(19) United States
(12) Patent Application Publication
(10) Pub. No.: US 2012/0029618 A1
(43) Pub. Date: Feb. 2, 2012

(54) Stent Connector Bump Design

(75) Inventors: Brian Tischler, New Brighton, MN (US); Dennis Peiffer, Brooklyn Park, MN (US); James F. Hemerick, Brooklyn Park, MN (US); Chad Perrin, Coon Rapids, MN (US)

(73) Assignee: BOSTON SCIENTIFIC SCIMED, INC., Maple Grove, MN (US)

(21) Appl. No.: 13/191,814

(22) Filed: Jul. 27, 2011

Related U.S. Application Data
(60) Provisional application No. 61/368,447, filed on Jul. 28, 2010.

(51) Int. Cl. A61F 2/82

(52) U.S. Cl. 623/1.16; 623/1.34; 623/1.2

(57) ABSTRACT

An endoprosthesis comprises a plurality of serpentine bands. Each serpentine band comprises a plurality of interconnected struts forming peaks and troughs. Adjacent serpentine bands are interconnected by one or more connectors. First and second serpentine bands are connected via at least one connector. The connector has a first side facing one the struts and a second side facing another of the struts. The connector extends from an inside of a trough of the first serpentine bands to the outside of a trough of the other of the serpentine bands. The connector has a first bump extending from the first side and a second bump extending from the second side opposite the first, the first bump located adjacent a first peak and the second bump located adjacent a second peak.

### CROSS-REFERENCE TO RELATED APPLICATIONS

This application claims the benefit of U.S. Provisional Application 61/368,447 filed Jul. 28, 2010, the entire contents of which is incorporated herein by reference.

### BACKGROUND OF THE INVENTION

A stent is a medical device which is introduced into a body lumen and is well known in the art. A stent is typically delivered in an unexpanded state to a desired location in a bodily lumen and then expanded by an internal radial force.

Stents, grafts, stent-grafts, vena cava filters, expandable frameworks, and similar implantable medical devices, are radially expandable endoprostheses, which are typically intravascular implants capable of being implanted transluminally and enlarged radially after being introduced percutaneously. Stents may be implanted in a variety of bodily lumens or vessels such as within the vascular system, urinary tracts, bile ducts, fallopian tubes, coronary vessels, secondary vessels, etc. Stents can be balloon-expandable, self-expanding or a combination of self-expanding and balloon-expandable (or “hybrid expandable”).

### SUMMARY OF THE INVENTION

The invention is directed to an endoprosthesis comprising a plurality of serpentine bands including a first serpentine band and a second serpentine band. Each serpentine band comprises a plurality of interconnected struts forming peaks and troughs. Adjacent serpentine bands interconnected by one or more connectors. The first and second serpentine bands are connected via at least one connector. The connector has a first side facing one of the struts and a second side facing another of the struts. The connector extends from an inside of a trough of the first serpentine bands to the outside of a trough of the other serpentine band. The connector has a first bump extending from the first side and a second bump extending from the second side opposite the first. The first bump is located adjacent a first peak and the second bump located adjacent a second peak.

Desirably, the endosprosthesis is in the form of a stent.

Desirably, the stent will comprise a plurality of connectors. Typically, each of the connectors will extend from an inside of a trough of one of the serpentine bands to the outside of a trough of the other the other of the serpentine bands. Some, and desirably all, of the connectors will have a first bump extending from the first side and a second bump extending from the second side opposite the first. The first bump may be located adjacent a first trough and the second bump located adjacent a second trough.

The stent may comprise a plurality of the serpentine bands and, desirably, three or more of the serpentine bands where adjacent serpentine bands are connected by a plurality of said connectors, each of which has a said first bump and a said second bump extending therefrom. Each connector may extend from a trough connected to a first adjacent strut via a first curved portion of the trough and connected to a second adjacent strut via a second curved portion of the trough, the first curved portion characterized by a first radius of curvature and the second curved portion characterized by a second radius of curvature. For each connector, the first bump typically extends from the connector and is characterized by a width as measured in a circumferential direction about equal to the first radius of curvature and the second bump typically extends from the connector and is characterized by a width as measured in a circumferential direction about to the second radius of curvature.

The invention is also directed to a self-expanding endoprosthesis, desirably, in the form of a stent, comprising a plurality of serpentine bands. Each serpentine band comprises a plurality of struts interconnected by peaks and troughs. Connectors extend between and connect adjacent serpentine bands. Each connector has a first bump extending from a first side toward an end of an adjacent strut and a second bump extending from a second side toward an end of another adjacent strut. The first and second bumps each have a width which is approximately equal to the separation between the connector and the adjacent strut when the endoprosthesis is in a crimped state.

Typically, the struts will be straight and each of the bumps will have a flat edge facing an adjacent strut. The flat edge will extend parallel to the connector. In lieu of flat edges, some or all of the bumps may have a curved edge extending non-parallel to the connector.

The connectors may extend from a trough on one serpentine band to a trough on an adjacent serpentine band.

The invention is also directed to a self-expanding endoprosthesis, desirably, in the form of a stent, comprising a plurality of serpentine bands. Each serpentine band comprises a plurality of interconnected strut pairs. Each strut pair has a first strut and a second strut adjacent the first strut. The first strut is connected to the second strut to form a peak, and a first strut pair is connected to a second strut pair to form a trough. Each first strut of the strut pair has a bump extending from a first side of the first strut toward a second side of the second strut. The bump has a width which is approximately equal to a distance between the first strut and the second strut. One or more connectors extend between and connect adjacent serpentine bands.

Typically, the struts will be straight and each bump will have a flat edge facing the second side of the second strut. The flat edge extends parallel to the first strut. In lieu of flat edges, some or all of the bumps may have a curved edge extending non-parallel to the second strut.

The connectors may extend from a peak on one serpentine band to a trough on an adjacent serpentine band. In some embodiments, the peak is circumferentially and longitudinally offset from the trough.

The invention is also directed to a stent delivery system comprising: a delivery catheter and a stent disposed about the delivery catheter and deployed therefrom. The stent comprises a plurality of serpentine bands including a first serpentine band and a second serpentine band. Each serpentine band comprises a plurality of interconnected struts forming peaks and troughs. Adjacent serpentine bands are interconnected by one or more connectors. The first and second serpentine bands are connected via at least one connector. The connector has a first side facing one the struts and a second side facing another of the struts. The connector extends from an inside of a trough of the first serpentine bands to the outside of a trough of the other of the serpentine bands. The connector has a first bump extending from the first side and a second bump extending from the second side opposite the first. The first bump is located adjacent a first peak and the second bump located adjacent a second peak. The first bump contacts a first strut and the second bump contacts a second strut.

The angle between each pair of interconnected struts that is not associated with a connector may be constant. The stent may be configured such that when the stent is deployed, the angle between each pair of interconnected struts that is not associated with a connector is also constant. Typically, at least the first bump of the connector will be located towards an end of the connector.

In at least one embodiment of the invention, the stent comprises a plurality of serpentine bands each having alternating peaks and troughs connected by straight struts. Two of said serpentine bands are connected one to the other via straight connectors which extend parallel to the longitudinal axis of the stent. Each straight connector extends from a trough of one serpentine band to a trough of an adjacent serpentine band. Each straight connector includes a first side and a second side that each extend from an outer wall surface of the stent to an inner wall surface of the stent. Each connector has a bumped out region of greater width than the remainder of the connector. The bumped out region is located between two circumferentially adjacent peaks and one end of the bumped out region is substantially aligned with the two circumferentially adjacent peaks. The first and second sides of the connector each having a bump thereon in the bumped out region of the connector. In at least one embodiment, each serpentine band has twenty-four struts; each strut width is between about 0.0028 in. and 0.0052 in.; each connector width is between about 0.0029 in. and 0.0053 in.; and each bumped out region is between about 0.0104 in and 0.0128 in. wide. In at least one embodiment, each serpentine band has three evenly spaced connectors. In at least one embodiment, the outer diameter of the stent is about 0.196 in. In at least one embodiment, the strut width is about 0.0040 in., the connector width is about 0.0041 in, the bumped out region is 0.0116 in. wide, and the wall thickness is 0.0076 in. In at least one embodiment, the stent is sized to fit within a 6 French catheter having an inner diameter of about 0.070 in.

### BRIEF DESCRIPTION OF THE DRAWINGS

FIG. 1 shows a partial view of an embodiment of the stent of the present invention.

FIG. 2A is a schematic showing of a stent similar to that of FIG. 1 on a delivery catheter.

FIG. 2B is a schematic showing the delivery system of FIG. 2A with the stent ready for deployment.

FIG. 2C is a schematic showing of a stent similar to that of FIG. 1 on a delivery catheter.

FIG. 2D is a schematic showing the delivery system of FIG. 2C with the stent partially deployed.

FIGS. 3A-3B show a prior art stent during expansion of the stent in the lumen.

FIGS. 4A-4B show an embodiment of the stent of the present invention during expansion in the lumen.

FIG. 5 shows a partial view of an embodiment of the invention.

FIG. 6 shows a partial view of an embodiment of the invention.

FIG. 7 shows a partial view of an embodiment of the invention.

FIG. 8 shows a partial view of an embodiment of the invention.

FIG. 9 shows a flattened view of an embodiment of the stent of the present invention.

### DESCRIPTION OF THE SPECIFIC EMBODIMENTS

While this invention may be embodied in many different forms, there are described in detail herein specific embodiments of the invention. This description is an exemplification of the principles of the invention and is not intended to limit the invention to the particular embodiments illustrated.

For the purposes of this invention, the term ‘inner surface’ shall refer to the surface of the stent or tubular member which faces the lumen of the stent or tubular member. The term ‘outer surface’ shall refer to the surface of the stent or tubular member which faces away from the lumen. The term ‘sidewall surface’ shall refer to the surface of the stent or tubular member which extends between the outer surface and the inner surface.

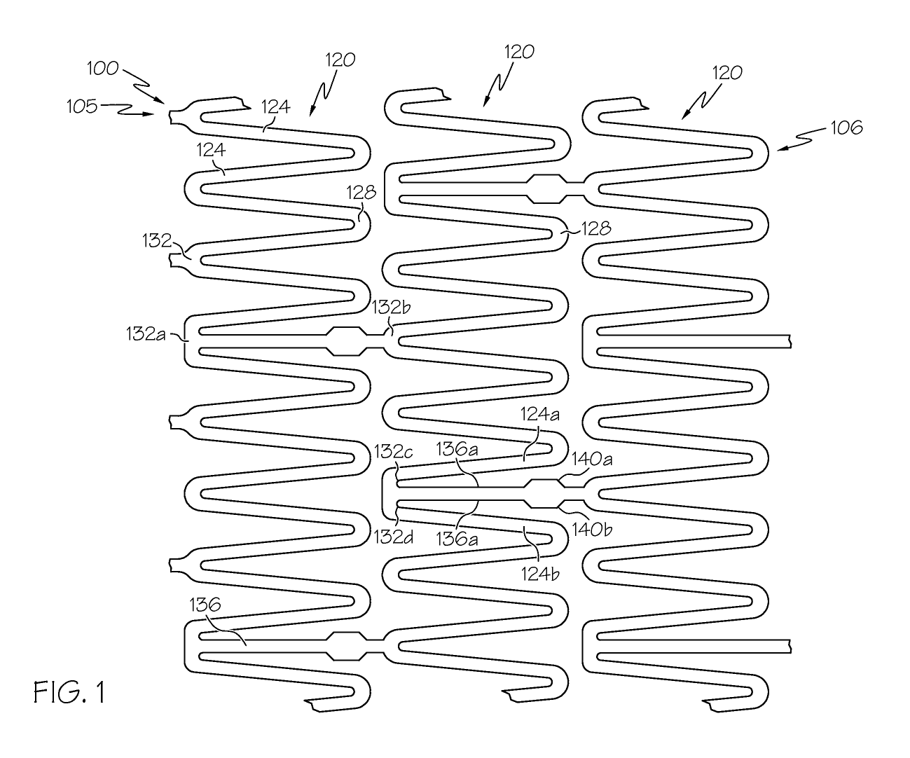

FIG. 1 shows a partial view of an endoprosthesis in the form of a stent, indicated generally at 100, in the unexpanded state. Stent 100 may be any kind of expandable prostheses such as a stent, stent-graft or graft. Stent 100 is a frame work formed of one or more interconnected serpentine bands 120. Stent 100 extends from a proximal end 105 to a distal end 106. Individual serpentine bands 120 comprise a plurality of interconnected struts 124. The serpentine band 120 of FIG. 1 is depicted with 16 struts. It is within the scope of the invention for a serpentine band to have fewer or more struts.

As shown in FIG. 1, struts 124 are desirably straight. The struts may, however, include curvature. In the case where the struts include curvature, the struts may be fundamentally straight with curved sides or the struts may be fundamentally curved. Where the strut is fundamentally straight with curved sides, a straight midline extends along the length of the strut and the entirety of the midline lies on the strut midway between the sides of the strut. Where the strut is fundamentally curved, the strut does not have a straight midline which extends along the length of the strut, the entirety of which lies on the strut midway between the sides of the strut.

Adjacent struts 124 are connected one to the other via a plurality of turns of serpentine band 120. For convenience, these turns may be referred to herein as peaks 128 and troughs 132. For the purposes of this disclosure, peaks 128 are defined to be located at a distal end 106 of serpentine band 120, while troughs 132 are defined to be located at a proximal end 105 of a serpentine band 120.

Adjacent serpentine bands 120 are connected to one another via connectors 136. As shown in FIG. 1, connector 136 extends from an inside portion 132a of a trough 132 to an outside portion 132b of another trough 132 of an adjacent serpentine band. Other arrangements of the connectors are also within the scope of the invention. For example, the connectors may extend from a peak of a serpentine band to a trough of an adjacent serpentine band. The connectors may also extend from locations along a serpentine band displaced from the peaks and troughs. To that end, the connectors may extend from locations along the serpentine band midway between a peak and a trough. They may extend from locations offset from the peak and trough as well as from the midway point between a peak and trough.

Typically, each connector 136 extends from trough 132 and connects to a first adjacent strut 124a via a first curved portion 132c of trough 132 and to a second adjacent strut 124b via a second curved portion 132d of trough 132. The first curved portion of the trough may be characterized by a first radius of curvature and the second curved portion of the trough may be characterized by a second radius of curvature.

Connectors 136 may be straight or curved. The ends of the connectors may be longitudinally and circumferentially aligned with one another or the ends may be longitudinally and circumferentially offset from one another. Where the connectors include curvature, the curvature may be similar to the curvature discussed above for the struts. As such, curved connectors may be fundamentally straight with curved sides or they may be fundamentally curved.

Stent 100 of FIG. 1 is shown with two connectors 136 between some serpentine bands 120 and one connector 136 between other serpentine bands 120. It is within the scope of the invention, however, for the stent to be provided with more connectors or fewer connectors. Typically, at least one connector will be provided between adjacent serpentine bands. The invention does contemplate, however, the possibility of some adjacent serpentine bands being connected one to the other directly without a separate connector.

As further shown in FIG. 1, connector 136 has a first side 136a which faces one adjacent strut 124a and a second side 136b which faces another adjacent strut 124b. The first and second sides 136a, 136b of connector 136 are opposite one another. Connector 136 has a first bump 140a extending from the first side 136a and a second bump 140b extending from the second side 136b. Bump 140a is desirably located adjacent a first peak and second bump 140b is desirably located adjacent a second peak.

Typically, bumps 140a and 140b will be of the same shape and size and will be mirror images of one another. It is also within the scope of the invention for the bumps on either side of a connector to be of different sizes and/or shapes. Desirably, the sidewall surface of the bumps will be complementary to the sidewall surface of the struts facing the bumps. Where the sidewall surface of the struts facing the bumps is straight, it is desirable for the sidewall surface of the bumps to include a straight portion which will contact the sidewall surface of the struts.

Also desirably, the first bump extending from the first side of a connector is characterized by a width, as measured in a circumferential direction, equal to the first radius of curvature of the first curved portion of the peak. The second bump extending from the second side of the connector is desirably characterized by a width, as measured in a circumferential direction, about equal to the second radius of curvature of the second curved portion of the peak.

Typically, the inventive stents will comprise a plurality of the serpentine bands. Adjacent serpentine bands will be connected by one or more of the connectors. Desirably at least one connector will include the above-described bumps. More desirably, all of the connectors will include the above-described bumps.

The invention is also directed to a stent or prosthesis comprising a plurality of serpentine bands, each of which comprises a plurality of struts interconnected by peaks and troughs. A plurality of connectors extends between and connects adjacent serpentine bands. Each connector has a first bump extending from a first side toward an end of an adjacent strut and a second bump extending from a second side toward an end of another adjacent strut. The first and second bumps each have a width which is approximately equal to the separation between the connector and the adjacent strut. Desirably, the struts are straight and each of the bumps has a flat edge facing an adjacent strut. The flat edge extends parallel to the connector. It is also within the scope of the invention for the struts to be straight and for each of the bumps to have a curved edge extending non-parallel to the connector. Typically, each connector extends from a peak on one serpentine band to a peak on an adjacent serpentine band.

The inventive stents disclosed herein may be provided in a balloon expandable form or in a self expanding form. They may also be provided in a hybrid form with both self expanding and the balloon expandable characteristics. The inventive stents, in many of the embodiments disclosed herein, may be deployed without the distortion that would result in the absence of the bumps or bumps disclosed herein.

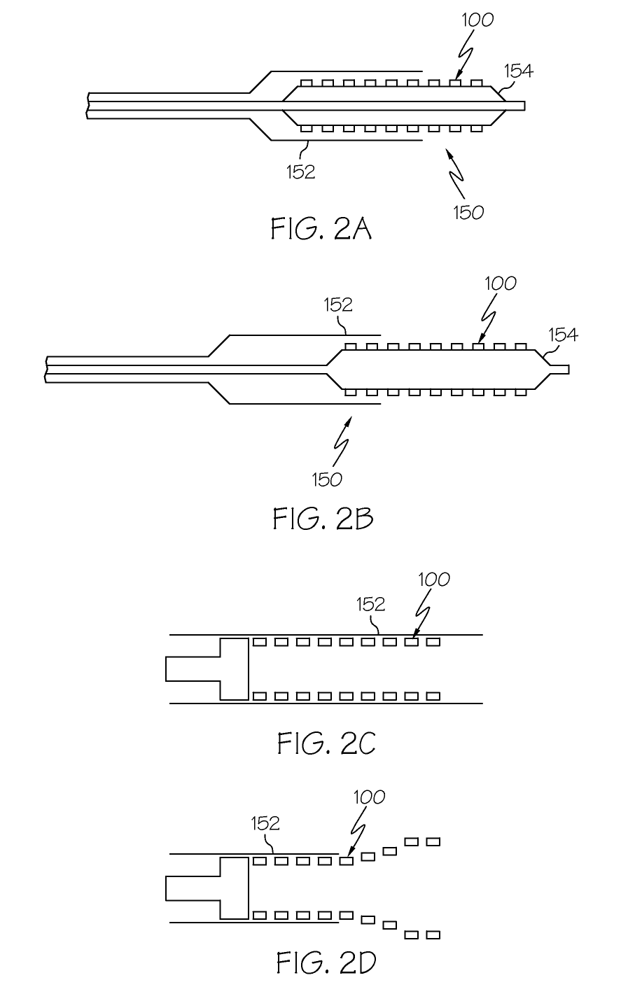

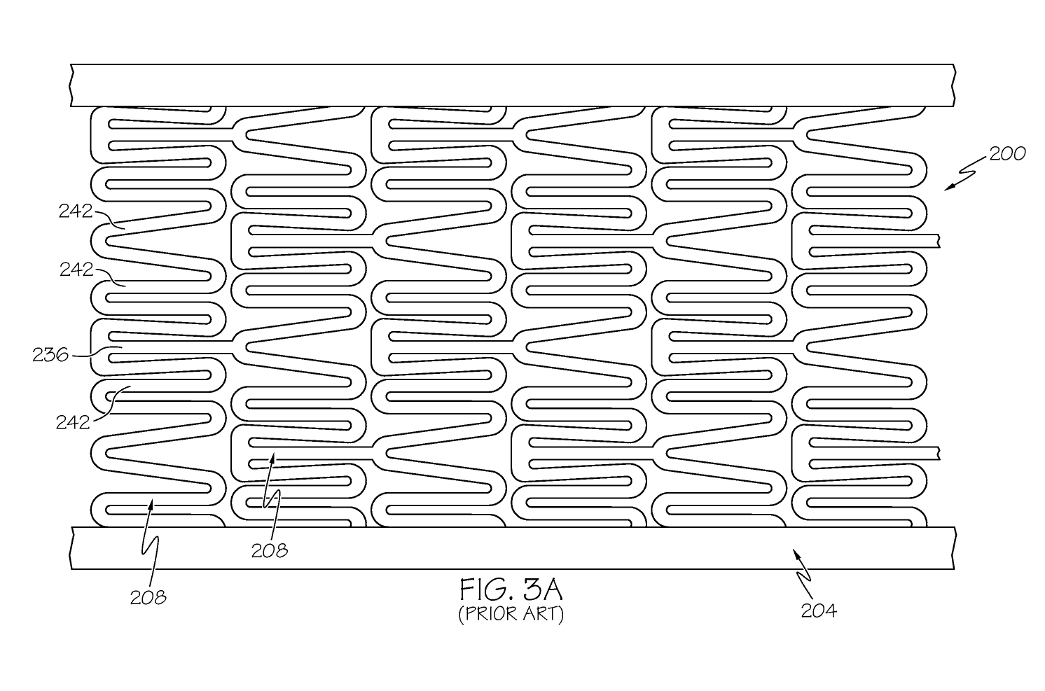

FIG. 2A shows an exemplary stent delivery system, which has stent 100 disposed about a delivery catheter 150. Delivery catheter 150 in this embodiment has a retractable sheath 152 that allows for self-expansion of the stent 100 in the lumen and a balloon 152 for balloon expansion. As shown in FIG. 2B, sheath 152 is retracted and balloon 154 can be expanded to deploy the stent in a bodily lumen.

FIG. 2C shows another exemplary stent delivery system, in which stent 100 is a self expanding stent disposed within a sheath 152. As shown in FIG. 2D, when the sheath 152 is retracted at the position where the stent is desired to be deployed, the stent 100 expands.

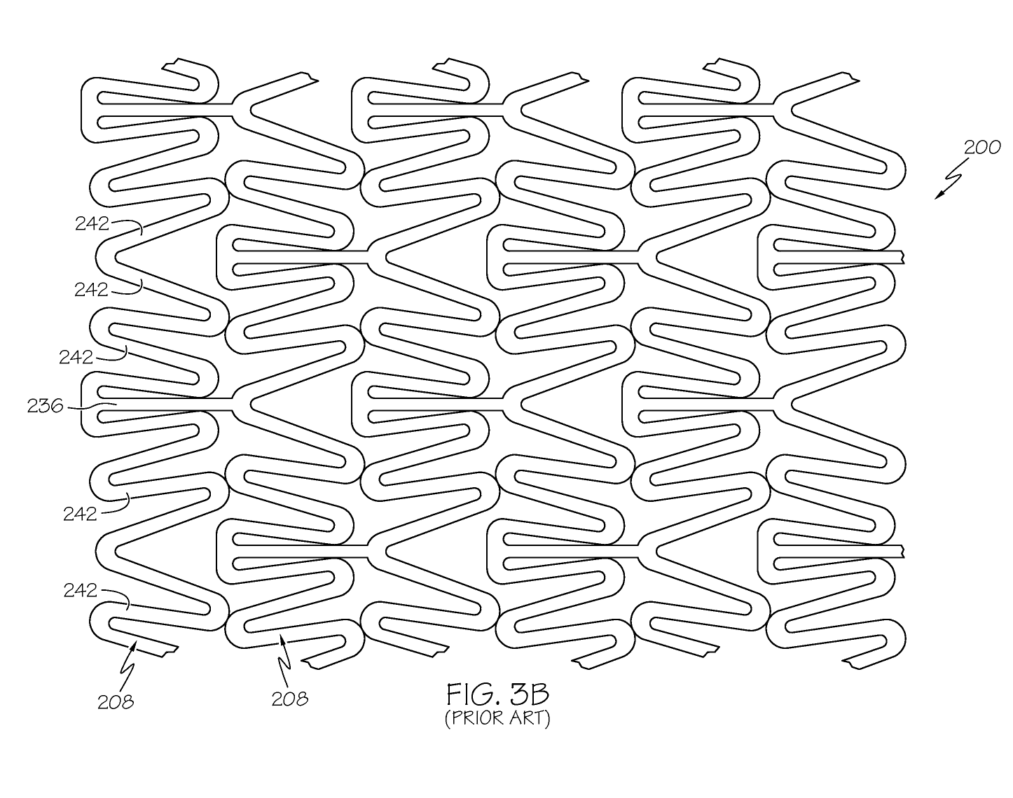

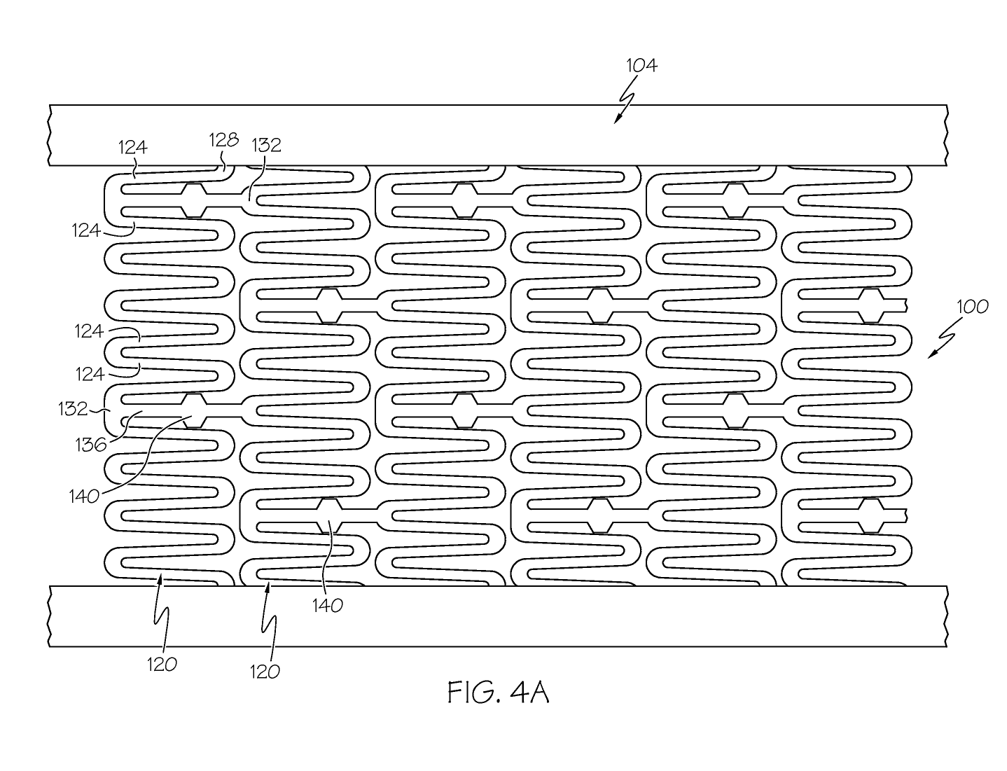

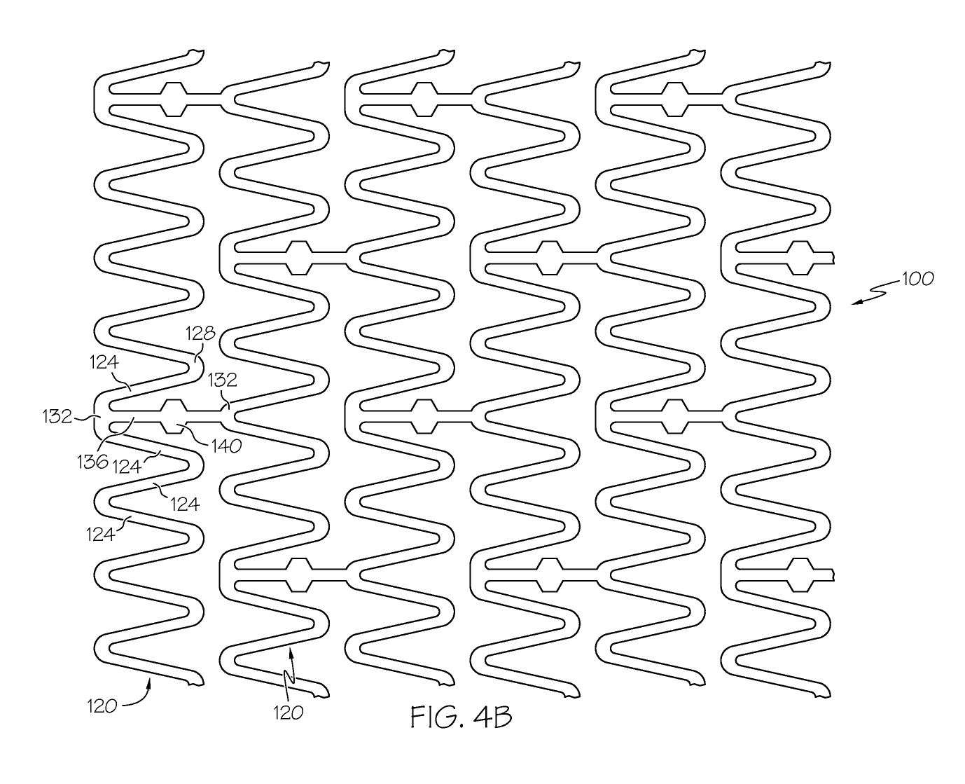

The inventive nature of one embodiment of the stent may be seen with reference to FIGS. 3A-3B and FIGS. 4A-4B, described further below.

FIGS. 3A-3B show a stent in the absence of bumps in a loaded state and a deployed state. In FIG. 3A, stent 200 is shown in a delivery catheter 204. The stent comprises a plurality of serpentine circumferential bands 208. Adjacent serpentine circumferential bands 208 are connected one to the other via straight connectors 236. Connectors 236 extend from the outside of one trough to the inside of another trough on an adjacent serpentine circumferential band. Each serpentine band comprises a plurality of struts 242. Some of the struts 242 are parallel to the longitudinal axis of stent 200. Those struts forming strut pairs with a connector 236 extending from the outside of the associated peak, as well as other struts, are disposed at an oblique angle relative to the longitudinal axis of stent 200. This distortion also results in other struts being disposed at oblique angles relative to the longitudinal axis of the stent.

When deployed, as shown in FIG. 3B, stent 200 has distortions in the strut angles throughout each circumferential band 208. The distortions are believed to result from axial force along connectors which results in differential openings of the turns and uneven expansion. Uneven expansion is believed to result from some struts, absent the bump, bending toward one another and others not bending. The stent will compress axially to a greater extent without bumps than with bumps. Reducing compression from both ends of the stent during delivery will affect deployed length. Compression may be reduced by providing one or more bumps which serve as a mechanical barrier to compression. The bumps take up the space that would have been filled by compression.

Rather than reducing the inner diameter of the sheath for the deployment mechanism, in the inventive stent shown in FIG. 4A, a bump 140 was added to the connector. The bump 140 is situated such that it contacts adjacent struts 124 such that the struts are parallel to one other to provide improved column strength in a constrained condition. The bump 140 is sized without significantly increasing surface area for metal to lumen ratio and drug compatibility concerns.

The deployment of an inventive stent is shown in FIGS. 4A-4B. In FIG. 4A, an inventive stent 100 with bumps 140 is shown in catheter 104. Circumferential bands 120 are shown prior to expansion. As shown in FIG. 4A, stent 100 does not have any of the distortions present in the stent of FIG. 3A. Stent 100 is shown deployed in FIG. 4C. The stent 100 does not have any of the irregular strut angles that are present in the prior art stent of FIGS. 3A-3B.

As shown in FIG. 4A, bump 140 is provided toward an end of connector 136. This location is particularly desirable in order to avoid the problem of “cowboy legging.” Specifically, if bump 140 is provided closer to a location midway between peak 128 and trough 132, the adjacent struts 124 may bend around the bump 140 when the stent is crimped. This phenomenon will not occur when the bump is positioned closer to the distal end of the connector. Nevertheless, in other embodiments of the invention, as shown below, the bump may be provided at other locations as well.

In some embodiments, when stent 100 is loaded on the catheter 104, bump 140 contacts adjacent struts 124. An angle between each pair of interconnected struts that is not associated with a connector is constant. In at least one embodiment, the struts of each pair of struts are in a substantially parallel configuration.

In some embodiments, when the stent 100 is deployed, as shown in FIG. 4B, the angle between each pair of interconnected struts that is not associated with a connector is constant. The stent 100 deploys evenly.

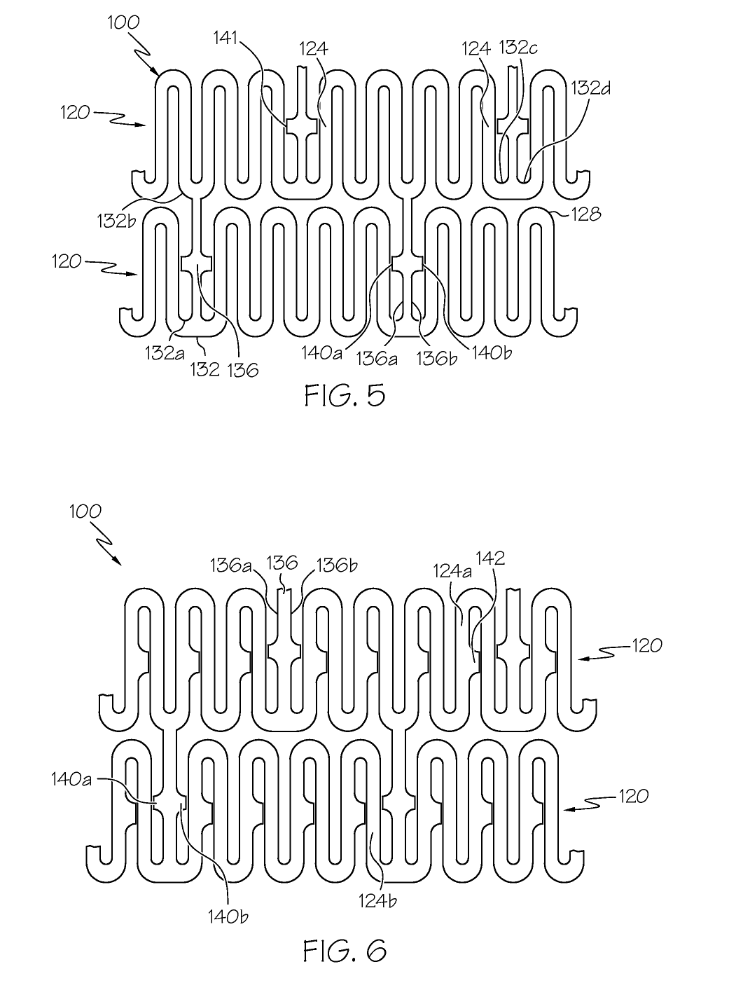

FIG. 5 shows a partial view of another embodiment of the invention in which the bump or bump 140a, 140b is provided along a connector 136 approximately midway between a peak and trough. Two serpentine bands 120 are shown in the embodiment of FIG. 5. The stent may include additional serpentine bands beyond the two shown in FIG. 5. The serpentine bands are interconnected by two connectors 136. Each connector 136 has at least one bump or bump 140 on a side of the connector. In the embodiment shown in FIG. 5, the bump or bump 140 has a trapezoidal shape with an outermost surface 141 of the bump being a flat surface. In the contracted (or loaded) state, bump 140 (particularly sidewall surface 141) abuts an adjacent strut 124 and is flush with the adjacent strut 124. While FIG. 5 shows a gap between bump 140, desirably at least a portion of the bump 140 will contact the adjacent strut in the contracted state. In at least one embodiment, the entire sidewall surface 141 contacts the adjacent strut. The trapezoidal shape of the bump 140 shown in FIG. 5 also allows for easy measurement of the width of the bump 140. While FIG. 5 shows the bump 140 having a trapezoidal shape, it is within the scope of the invention for the bumps 140 to have other configurations.

FIG. 6 shows a partial view of another embodiment of the invention in which bumps 142 are provided along struts 124a of a serpentine band in addition to bumps 140a and 140b along sides 136a and 136b of connectors 136. The serpentine bands may also include struts 124b with bumps.

Desirably, as shown in FIG. 6, the bumps are provided approximately midway between a peak 128 and trough 132. The connectors 136 between the adjacent serpentine bands 120 are shown extending axially relative to the longitudinal axis of the stent. It is within the scope of the invention for the connectors to be parallel to the longitudinal axis and/or for the connectors to include curvature. The stent may include additional serpentine 120 bands beyond the two shown in the figure.

In the serpentine bands 120 shown in FIG. 6, every other strut 124 includes a bump 142. Thus, the serpentine band has an alternating pattern of straight struts including bumps (struts 124a) and straight struts lacking bumps (struts 124b). The serpentine band may include additional bumps. For example, each strut 124 may include a bump. Also, fewer struts may include bumps. For example, every third, fourth, fifth or sixth strut may include such a bump.

Where one or more struts of a serpentine band are provided with a bump, the connector connecting the serpentine band to an adjacent serpentine band may have a bump, as shown in FIG. 6 or may lack a bump. Where the connector 136 is provided with a bump 140 and an adjacent strut 124 has a bump 142, as shown in FIG. 6, bump 140 is slightly offset from bump 142. It is within the scope of the invention for the bumps on the connector to be aligned with the bumps on the adjacent strut.

While FIG. 6 shows a slight gap between bump 140a, 140b and an adjacent strut 124 and a slight gap between bump 142 and an adjacent strut 124, desirably at least a portion of the bumps 140, 142 abut the surface of the adjacent strut 124 in the contracted state. In at least one embodiment, the entire surface of the bump 140, 142 abuts the surface of the adjacent strut 124 in the contracted state.

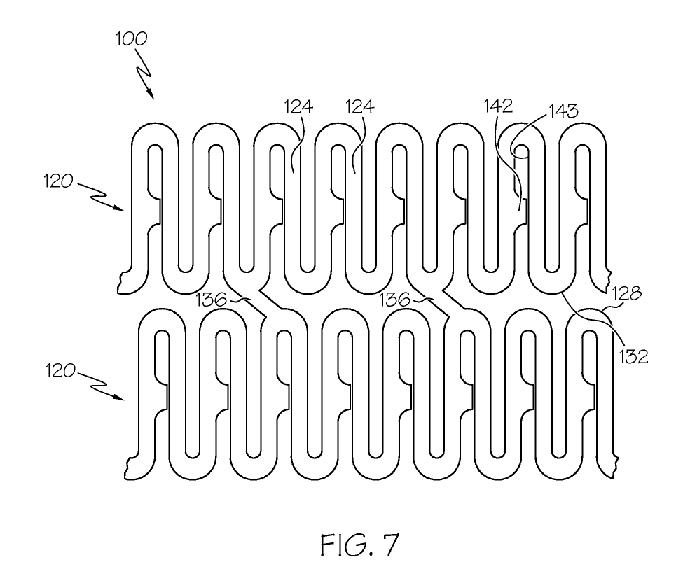

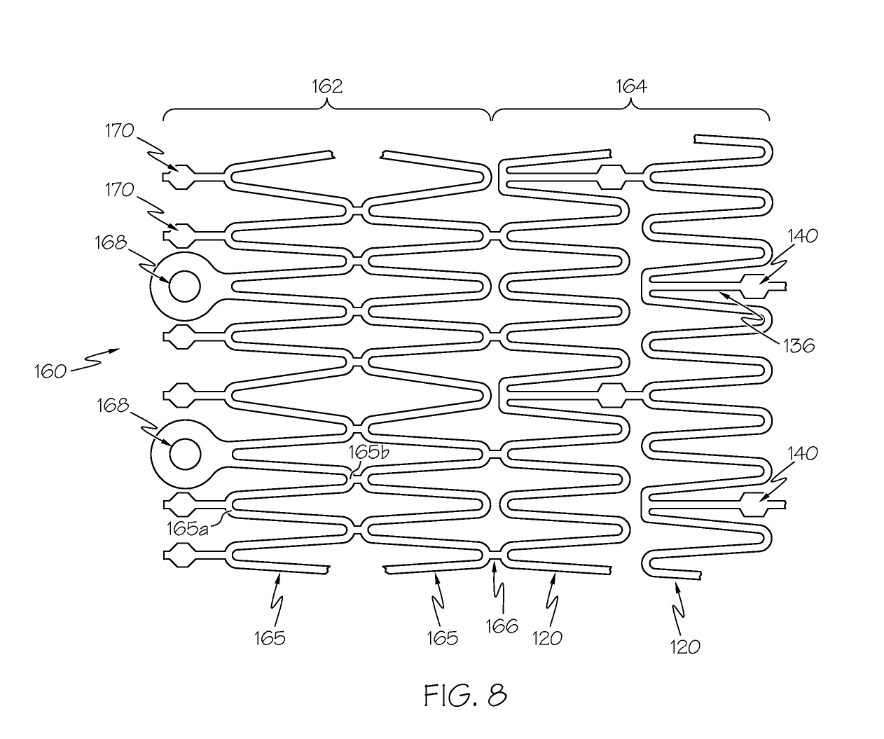

FIG. 7 shows a partial view of another embodiment of the invention in which bumps 142 are provided only along some of the struts of the serpentine bands, and connectors 136 do not have bumps. As shown in FIG. 7, the bumps 142 are provided along the struts 124 approximately midway between a peak 128 and a trough 132. Every other strut is provided with a bump. In the embodiment shown in FIG. 7, connectors 136 connect a peak 128 of a first serpentine band 120 with a longitudinally and circumferentially offset trough 132 of an adjacent serpentine band 120. Connectors 136 extend at an oblique angle relative to the longitudinal axis of the stent. The stent may include additional serpentine bands beyond the two shown in the figure. The serpentine bands may be arranged such that the connectors extend between longitudinally aligned peaks and troughs. The connectors may be straight or may include curvature.

While FIG. 7 shows a slight gap between each bump 142 and an adjacent strut 124, desirably at least a portion of the bumps abut the surface of the adjacent strut in the contracted state. In at least one embodiment, the entire surface of the bump abuts the surface of the adjacent strut in the contracted state.

FIG. 8 shows a partial view of another embodiment of the invention, which has bump extensions between radiopaque marker housings at an end of the stent. At a first end 160 of the stent of this embodiment is first region 162, which is connected to a second region 164.

First region 162 comprises at least one serpentine band 165 (two serpentine bands are shown in FIG. 8) having a plurality of interconnected strut pairs. Each strut pair has a first strut and a second strut adjacent the first strut. The first strut is connected to the second strut to form a peak 165a and a first strut pair is connected to a second strut pair to form a trough 165b. A serpentine band 165 is connected to an adjacent serpentine band 165 by a connector 166. In some embodiments, the serpentine band 165 is connected to an adjacent serpentine band 165 by a connector 166 at trough 165b.

At an end 160 of first region 162 are a plurality of radiopaque markers 168. Each radiopaque marker 168 is attached to a peak 165a of the endmost serpentine band 165 and extends from the peak towards the end 160 of the stent. A bump extension 170 extends from each of a plurality of peaks 165a which are not associated with a radiopaque marker 168. The bump extension 170 also extends from the peak towards the end 160 of the stent. In at least the embodiment shown, the bump extensions 170 are between radiopaque markers 168 along the circumference of the stent.

In at least the embodiment shown in FIG. 8, the bump portion 172 has a trapezoidal shape with a stub 174 protruding from the bump portion. A member 176 joins the bump portion 172 to the peak 165a. In other embodiments, the bump extension 170 can have a circular shape, does not have a stub, and may have other configurations. Using these bump extension 170 around radiopaque marker housings 168 ensures equal spacing of the markers 168 and provides extra strength or rigidity to the end of the stent when the stent is deployed.

Second region 164 of the embodiment shown in FIG. 8 has a plurality of serpentine bands 120 as previously described herein. Each serpentine band is connected to an adjacent serpentine band in the second region 164 with a plurality of connectors 136 having bumps 140. Second region 164 is connected to first region 162 by connectors 166. In at least one embodiment connector 166 connects a trough 165b of the first region 162 with a confronting peak of an adjacent serpentine band 120 of the second region 164. While in the embodiment shown in FIG. 8, the serpentine bands of the first region 162 are not the same as the serpentine bands of the second region 164, it is within the scope of this invention that the serpentine bands of first region 162 are the same as the serpentine bands 120 the second region 164. In some embodiments, the serpentine bands 165 of the first region 162 may also be connected to one another by connectors similar to connectors 136. In some embodiments, these connectors can also have bumps similar to bumps 140.

In some embodiments of the invention, the stent, or portion thereof, may be provided with a substance. The substance may be a coating or a portion of the stent constructed and arranged to deliver the substance to a location in a body lumen. The substance may be a drug, genetic material, cells, a non-genetic therapeutic agent, a polymer matrix having a therapeutic component or any other substance which it would desirable to deliver into a body lumen. In some embodiments the substance 18 may be a coating of SIBS (styrene isobutylene styrene); polycarboxylic acids; cellulosic polymers, including cellulose acetate and cellulose nitrate; gelatin, polyvinylpyrrolidone; cross-linked polyvinylpyrrolidone; polyanhydrides including maleic anhydride polymers; polyamides; polyvinyl alcohols; copolymers of vinyl monomers such as EVA; polyvinyl ethers; polyvinyl aromatics; polyethylene oxides; glycosaminoglycans; polysaccharides; polyesters including polyethylene terephthalate; polyacrylamides; polyethers; polyether sulfone; polycarbonate; polyalkylenes including polypropylene, polyethylene and high molecular weight polyethylene; halogenated polyalkylenes including polytetrafluoroethylene; polyurethanes; polyorthoesters; proteins; polypeptides; silicones; siloxane polymers; polylactic acid; polyglycolic acid; polycaprolactone; polyhydroxybutyrate valerate and blends and copolymers thereof; PLGA, coatings from polymer dispersions such as polyurethane dispersions (BAYHDROL.RTM., etc.); fibrin; collagen and derivatives thereof; polysaccharides such as celluloses, starches, dextrans, alginates and derivatives; hyaluronic acid; squalene emulsions; polyacrylic acid, available as HYDROPLUS™ from Boston Scientific Corporation, Natick, Mass., and described in U.S. Pat. No. 5,091,205, the entire contents of which are hereby incorporated herein by reference.

The substance may be disposed on any of the surfaces of the stent or within holes in the stent surface using any known technique for doing so. The bumps may be placed in any manner along a given connector or connectors and/or along any strut or struts. The individual bumps may be provided with a wide range of shapes, sizes, configurations, and compositions. Desirably, the side of the bump facing the adjacent strut (or connector) will have a shape which is complementary to that of the adjacent strut.

Bumps 140, 142 and bump extensions 170 may be constructed from any material desired and, desirably, of a biocompatible material or materials. Where the bump includes a non-biocompatible material in its construction, the bump preferably includes a biocompatible coating. More desirably, the bumper is constructed out of the same material as the connector or strut which the bump extends from. The bump may be an inherent part of the connector or strut, being merely a bump of connector or strut material, or it may be a separate component which is welded, adhered, or otherwise engaged to the connector or strut.

Depending on the flexibility of the stent as well as of the catheter, the bump in the various embodiments of the present invention may be provided with a wide range of heights or thicknesses relative to the connector or strut from which the bump extends. The bump may extend from about 0.0002 inches to about 0.015 inches from a given connector or strut. Preferably, the bump extends from a connector or strut by about 0.0002 to about 0.015 inches. In general, for a stent whose struts are from 0.002 inches to about 0.006 inches wide and connectors are from 0.002 inches to about 0.006 inches wide, the bump width will be approximately from 0.002 inches to about 0.006 inches wide. In other words, in at least one embodiment, the strut width will be the same as the connector width, and the connector width will be the same as the bump width.

The invention is also directed to a medical device delivery system here comprising a catheter and any the inventive stents disclosed herein. Where the stent is balloon expandable, the system will typically comprise a balloon catheter and a balloon expandable stent, as disclosed herein, disposed about the balloon catheter. In the case of a self-expanding stent, the system will typically include a catheter about which the self-expanding stent is disposed as well as a retractable sheath or cover disposed over the stent. Portions of the catheter may include any of the coatings disclosed herein.

The invention is also directed to a stent delivery system comprising a delivery catheter and a stent disposed about the delivery catheter and deployed therefrom. The stent comprises a plurality of serpentine bands including a first serpentine band and a second serpentine band. Each serpentine band comprises a plurality of interconnected struts forming peaks and troughs. Adjacent serpentine bands are interconnected by one or more connectors. The first and second serpentine bands are connected via at least one connector. The connector has a first side facing one the struts and a second side facing another of the struts. The connector extends from an inside of a trough of the first serpentine bands to the outside of a trough of the other of the serpentine bands. The connector has a first bump extending from the first side and a second bump extending from the second side opposite the first. The first bump is located adjacent a first peak and the second bump located adjacent a second peak. The first bump contacts a first strut and the second bump contacts a second strut.

The angle between each pair of interconnected struts that is not associated with a connector may be constant. The stent may be configured such that when the stent is deployed, the angle between each pair of interconnected struts that is not associated with a connector is also constant. Typically, at least the first bump of the connector will be located towards an end of the connector.

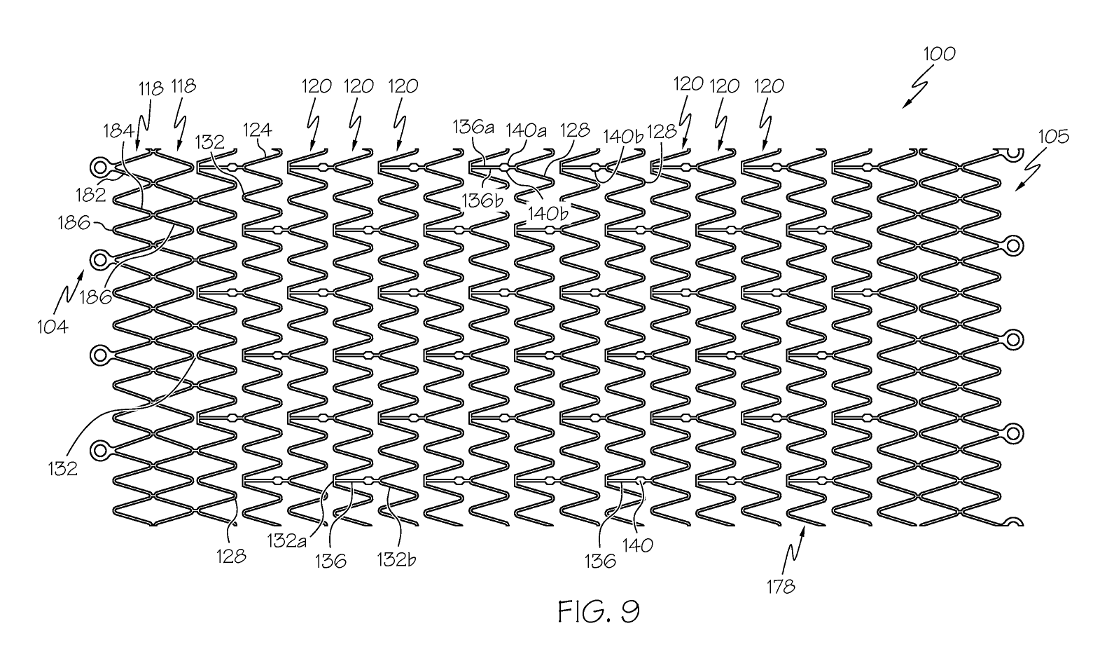

FIG. 9 shows a flat view of an embodiment of the present invention. As shown, the stent comprises a plurality of serpentine bands 120, each serpentine band 120 having alternating peaks 128 and troughs 132 connected by struts 124. The struts 124 are shown as straight struts in the embodiment, but other configurations are within the scope of the invention. Two of said serpentine bands are connected one to the other via straight connectors 136 which extend parallel to the longitudinal axis. Each connector 136 extends from a trough 132 of one serpentine band to a trough 132 of an adjacent serpentine band. Each straight connector has a first side 136a and a second side 136b that each extend from an outer wall surface 178 of the stent to an inner wall surface of the stent (not shown), forming the wall thickness of the stent. Each connector 136 has a bumped out region 180 of greater width than the remainder of the connector 136, the first and second sides 136a, 136b each having a bump 140 thereon in the bumped out region 180 of the connector 136. The bumped out region is located between two circumferentially adjacent peaks 128 and one end of the bumped out region 140 is substantially aligned with the two circumferentially adjacent peaks 128.

The stent in FIG. 9 also has a pair of serpentine bands 181 at both the proximal end 104 and the distal end 105 of the stent. Each serpentine band 181 comprises a plurality of struts 182 forming alternating peaks 184 and troughs 186. For the purposes of this disclosure, peaks 184 are defined to be located at a distal end 105 of serpentine band 120, while troughs 186 are defined to be located at a proximal end 106 of a serpentine band 120. The serpentine bands 181 are connected to each other where a peak 184 of the first serpentine band 181 confronts a trough 186 of the adjacent serpentine band 181. In at least the embodiment shown, the first serpentine band 181 is connected to the adjacent serpentine band 181 at every peak 184 of the first serpentine band 181. In the embodiment shown, serpentine band 181 is connected to some of the troughs 132 of an adjacent serpentine band 120, but never at any of the troughs that are connected to connectors 136. Other configurations are within the scope of the invention.

The embodiment shown in FIG. 9 is for a stent having an outer diameter of 0.196 inches (5 mm) and is sized to fit within a 6 French catheter.

In this embodiment, each serpentine band 120 has twenty-four struts 124. Each strut width is between about 0.0028 inches and 0.0052 inches. The width of each connector 136 is between about 0.0029 inches and 0.0053 inches. Each bumped out region 140 is between about 0.0104 inches and 0.0128 inches wide. The wall thickness of the stent between the outer surface and the inner surface is between about 0.0061 inches and 0.0091 inches. In a particular embodiment, the strut width is about 0.0040 inches, the connector width is about 0.0041 in, the bumped out region is 0.0116 inches wide, and the wall thickness is 0.0076 inches.

In some embodiments, each serpentine band 120 has at least two evenly spaced connectors 136. In the embodiment shown in FIG. 9, each serpentine band 120 has three evenly spaced connectors 136.

When the stent of FIG. 9 is crimped, the struts 124 are parallel and the struts 124 touch the bumps 140. The stent of FIG. 9 may in some embodiments be self-expanding. The configuration of the bumps 140 on the connectors 136 allows the stent to expand more evenly from the crimped condition to the expanded condition.

In addition to being directed to the specific combinations of features claimed below, the invention is also directed to embodiments having other combinations of the dependent features claimed below and other combinations of the features described above.

In at least one embodiment, an endoprosthesis is self-expanding and comprises a plurality of serpentine bands, each band comprising a plurality of struts interconnected by peaks and troughs; and a plurality of connectors extending between and connecting adjacent serpentine bands, each connector having a first bump extending from a first side toward an end of an adjacent strut and a second bump extending from a second side toward an end of another adjacent strut. The first and second bumps each have a width which is approximately equal to the separation between the connector and the adjacent strut when the endoprosthesis is in a crimped state. In one embodiment, the endoprosthesis is a stent. The struts can be straight. At least one of the bumps has either a flat edge facing an adjacent strut and extending parallel to the connector or a curved edge extending non-parallel to the connector. In at least one embodiment, each connector extends from a trough on one serpentine band to a trough on an adjacent serpentine band.

In at least one embodiment, a stent delivery system is provided. The stent delivery system comprises a delivery catheter and a stent disposed about the delivery catheter and deployed therefrom. The stent comprises a plurality of serpentine bands including a first serpentine band and a second serpentine band, each serpentine band comprising a plurality of interconnected struts forming peaks and troughs, adjacent serpentine bands interconnected by one or more connectors, the first and second serpentine bands connected via at least one connector, the connector having a first side facing one the struts and a second side facing another of the struts, the connector extending from an inside of a trough of the first serpentine band to the outside of a trough of the second serpentine band, wherein the connector has a first bump extending from the first side and a second bump extending from the second side opposite the first, the first bump located adjacent a first peak and the second bump located adjacent a second peak, wherein the first bump contacts a first strut and the second bump contacts a second strut. In at least one embodiment, an angle between each pair of interconnected struts that is not associated with a connector is constant. In at least one embodiment, the stent is configured such that when the stent is deployed, the angle between each pair of interconnected struts that is not associated with a connector is constant. In at least one embodiment, the first bump of the connector is located towards an end of the connector.

In at least one embodiment, an endoprosthesis comprises a first region at a first end of the endoprosthesis, the first region comprising: one serpentine band comprising a plurality of interconnected strut pairs, each strut pair having a first strut and a second strut adjacent the first strut, the first strut connected to the second strut to form a peak, a first strut pair connected to a second strut pair to form a trough, and a plurality of extensions extending axially from peaks of the serpentine band towards the first end, wherein a first extension and a second extension comprise radiopaque markers and wherein extensions circumferentially between the first extension and the second extension comprise bumps.

In at least one embodiment, a self-expanding endoprosthesis comprises a plurality of serpentine bands, each band comprising a plurality of interconnected strut pairs, each strut pair having a first strut and a second strut adjacent the first strut, the first strut connected to the second strut to form a peak, a first strut pair connected to a second strut pair to form a trough, wherein each first strut has a bump extending from a first side of the first strut toward a second side of the second strut, wherein the bump has a width which is approximately equal to a distance between the first strut and the second strut; and at least one connector extending between and connecting adjacent serpentine bands. In at least one embodiment, the endoprosthesis is in the form of a stent. In at least one embodiment, the first and second struts of each strut pair are straight and the bump has a flat edge facing the second side of the second strut, the flat edge extending parallel to the second strut. In another embodiment, the struts are straight and each bump has a curved edge extending non-parallel to the second strut. In at least one embodiment, the endoprosthesis comprises a plurality of said connectors, wherein each connector extends from a peak on one serpentine band to a trough of an adjacent serpentine band. In at least one embodiment the peak is circumferentially and longitudinally offset from the trough.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. An endoprosthesis comprising a plurality of serpentine bands including a first serpentine band and a second serpentine band,
each serpentine band comprising a plurality of interconnected struts forming peaks and troughs, adjacent serpentine bands interconnected by one or more connectors
the first and second serpentine bands connected via at least one connector, the connector having a first side facing one the struts and a second side facing another of the struts,
the connector extending from an inside of a trough of the first serpentine bands to the outside of a trough of the other of the serpentine bands,
wherein the connector has a first bump extending from the first side and a second bump extending from the second side opposite the first, the first bump located adjacent a first peak and the second bump located adjacent a second peak.

2. The endoprosthesis of claim 1 in the form of a stent.

3. The endoprosthesis of claim 1, wherein the endoprosthesis is self-expanding.

4. The endoprosthesis of claim 1 comprising a plurality of said connectors, each of said connectors extending from an inside of a trough of one of the serpentine bands to the outside of a trough of the other the other of the serpentine bands, each of said connectors having a first bump extending from the first side and a second bump extending from the second side opposite the first, the first bump located adjacent a first peak and the second bump located adjacent a second peak.

5. The endoprosthesis of claim 4, comprising three of said serpentine bands, adjacent serpentine bands connected by a plurality of said connectors, each of which has a said first bump and a said second bump extending therefrom.

6. The endoprosthesis of claim 5, each connector extending from a trough connected to a first adjacent strut via a first curved portion of the trough and connected to a second adjacent strut via a second curved portion of the trough, the first curved portion characterized by a first radius of curvature and the second curved portion characterized by a second radius of curvature,
wherein for each connector, the first bump extending from the connector is characterized by a width as measured in a circumferential direction about equal to the first radius of curvature and the second bump extending from the connector is characterized by a width as measured in a circumferential direction about to the second radius of curvature.

7. An endoprosthesis comprising a plurality of serpentine bands, each serpentine band having alternating peaks and troughs connected by straight struts, the endoprosthesis including a section comprising two of said serpentine bands which are connected one to the other via straight connectors which extend parallel to the longitudinal axis, each straight connector within the section extending from a trough of one serpentine band to a trough of an adjacent serpentine band, each straight connector including a first side and a second side, the first and second sides extending from an outer wall surface of the endoprosthesis to an inner wall surface of the endoprosthesis, each connector having a bumped out region of greater width than the remainder of the connector, the first and second sides each having a bump thereon in the bumped out region of the connector, wherein the bumped out region is located between two circumferentially adjacent peaks and one end of the bumped out region is substantially aligned with the two circumferentially adjacent peaks.

8. The endoprosthesis of claim 7, wherein each serpentine band has three evenly spaced connectors.

9. The endoprosthesis of claim 7, wherein each serpentine band has twenty-four struts, each strut width being between about 0.0028 in. and 0.0052 in., each connector width being between about 0.0029 in. and 0.0053 in., each bumped out region being between about 0.0104 in and 0.0128 in. wide.

10. The endoprosthesis of claim 9, wherein the outer diameter of the endoprosthesis is about 0.196 in.

11. The endoprosthesis of claim 9, wherein the strut width is about 0.0040 in., the connector width is about 0.0041 in, the bumped out region is 0.0116 in. wide, and the wall thickness is 0.0076 in.

12. An endoprosthesis comprising:
a first region at a first end of the endoprosthesis, the first region comprising:
one serpentine band comprising a plurality of interconnected strut pairs, each strut pair having a first strut and a second strut adjacent the first strut, the first strut connected to the second strut to form a peak, a first strut pair connected to a second strut pair to form a trough,
a plurality of radiopaque markers, each radiopaque marker connected to a peak of the serpentine band, each radiopaque marker extending axially from a peak of the serpentine band towards the first end; and
at least one bump extension connected to a peak of the serpentine band that is not connected to the radiopaque marker, the at least one bump extension extending axially in the same direction as the radiopaque marker.

13. The endoprosthesis of claim 12, further comprising a second region interconnected with the first region, the second region comprising:
a plurality of serpentine bands, each band comprising a plurality of interconnected strut pairs, each strut pair having a first strut and a second strut adjacent the first strut, the first strut connected to the second strut to form a peak, a first strut pair connected to a second strut pair to form a trough; and
at least one connector extending between and connecting adjacent serpentine bands.

14. The endoprosthesis of claim 13, wherein at least one trough of a serpentine band of the first region is connected to at least one peak of an adjacent serpentine band of the second region by a connector.

15. The endoprosthesis of claim 13, wherein the at least one connector has a first bump extending from a first side toward an end of an adjacent strut and a second bump extending from a second side toward an end of another adjacent strut.

16. The self-expanding endoprosthesis of claim 12, wherein at least one bump extension is between at least two radiopaque markers along a circumference of the stent.

17. The self-expanding endoprosthesis of claim 12, wherein each first strut of the second region has a bump extending from a first side of the first strut of the second region toward a second side of the second strut of the second region, wherein the bump has a width which is approximately equal to a distance between the first strut of the second region and the second strut of the second region.
